(19) United States
(12) Patent Application Publication
(10) Pub. No.: US 2017/0273705 A1
(43) Pub. Date: Sep. 28, 2017

(54) SKIN TAG REMOVER

(71) Applicants: Mona Zallum, Grimsby (CA); Moe Alqebalie, Grimsbyc, CA (US); Anwar Alqebalie, Al-Khobar (SA)

(72) Inventors: Mona Zallum, Grimsby (CA); Moe Alqebalie, Grimsbyc, CA (US); Anwar Alqebalie, Al-Khobar (SA)

(21) Appl. No.: 15/471,066

(22) Filed: Mar. 28, 2017

Related U.S. Application Data
(60) Provisional application No. 62/314,032, filed on Mar. 28, 2016.

(51) Int. Cl. A61B 17/322; A61K 31/167; A61K 31/155; A61L 31/16; A61L 31/08

(52) U.S. Cl. CPC A61B 17/322; A61L 31/16; A61L 31/08; A61K 31/155; A61K 31/167; A61B 2017/00893

(57) ABSTRACT

The present invention is a skin tag remover which includes a cord formed into a loop at a tag end for placing around a skin tag, a cord housing for receiving a portion of the cord there through and a finger ring attached to a ring end of the cord. Pulling on the finger ring tightens the loop around the skin tag and places the cord into a constricted position so that blood flow is restricted to the skin tag. A blade is included in the cord housing with a plunger that urges the blade downward to sever and detach the cord from the cord housing.

This application claims priority from previously filed U.S. provisional patent application 62/314,032. filed Mar. 28, 2016 by Mona Zallum, Moe Alqebalie and Anwar Alqebalie under the title SKIN TAG REMOVER.

### FIELD OF THE INVENTION

The present concept relates to methods and devices of removing skin tags and more particularly relates to a portable and potentially disposable device for removing skin tags using a strangulation method.

### BACKGROUND OF THE INVENTION

Skin tags which are also known as acrochordons are small tumours or protrusions that form on an area of skin as a bulbous mass that may appear anywhere in the body including the neck, armpit, groin, face and eyelids. Skin tags vary in size but are usually between 1 mm and 50 mm in length. The surface of the skin tag may be smooth or regular in appearance and often the skin tag is raised from the surface of the skin on a fleshy stalk called a peduncle which is commonly referred to as the base of the skin tag.

Skin tags are by and large harmless however they are frequently removed for cosmetic reasons or due to irritation from contact with clothing or jewellery. Skin tags may also interfere with shaving and other grooming rituals and may appear on the skin in unwanted areas such as in facial regions.

There are known methods that are currently being used to remove skin tags including cauterization, electrolysis, cryo-surgery, ligation and excision. Typically these procedures are performed at a doctor's office, and may involve the use of a local anaesthetic, freezing with liquid nitrogen, burning by way of a high frequency electric current or excision using a scalpel or scissors.

There have been numerous attempts made to develop assistance for skin tag removal without the need to visit a doctor's office. In this regard there are examples described in U.S. patent application 2005/0165423 by Gallagher et al. filed on Jan. 23, 2004 and also in U.S. patent application 2002/0111640 to Krause et al. filed on Feb. 13, 2002 and also in U.S. patent application 2005/0149063 to Young et al. filed on Oct. 20, 2004.

However all of these devices require a certain amount of finger dexterity and an ability to manipulate very small items with the fingers in order to put or place the device in position.

There is a need for a skin tag removing device that is not only fast and effective in removing skin tags but can be applied easily without the use of medical personnel which is easy to deploy and can reliably and reproducibly effectuate skin tag removal without pain.

### SUMMARY OF THE INVENTION

A skin tag remover which includes:
a) A cord formed into a loop at a tag end, the loop for placing around a skin tag;
b) A cord housing for receiving a portion of the cord there through;
c) A finger ring attached to a ring end of the cord; such that pulling on the finger ring tightens the loop around the skin tag and places the cord into a constricted position;
d) A means for locking the cord in a constricted position.

Preferably further including a means for cutting the cord such that the loop is separated from the finger ring.

Preferably wherein the locking means includes a lock housing slide-able received within a lock receptacle, such that the cord lock is slideably detachable from the cord housing.

Preferably wherein the locking means includes lock fingers which abut against the cord protrusions allowing movement of the cord in only the constricting direction thereby preventing the cord from loosening from around the skin tag.

Preferably wherein the cutting means includes:
a) A blade biased in a normal open position above the cord;
b) A plunger connected to the blade for urging downwardly with finger pressure thereby cutting the cord;
c) The plunger received within a plunger cavity defined within the cord housing.

Preferably wherein the cutting means includes a blade receiving guide for receiving the blade therein in a closed position.

Preferably wherein the cord housing includes a cord channel guiding the cord through the cord housing.

Preferably wherein the cord housing includes a semi-circular ring receiving end and a semi-circular loop receiving end for mounting the loop and finger ring in position when placed on the skin.

Preferably wherein the cord is selected from among a ball cord, a stepped cord and a barbed cord.

Preferably wherein the cord is coated with an antibacterial agent to prevent infection.

Preferably wherein antibacterial coating is chlorhexidine.

Preferably wherein the cord is further coated with a local anaesthetic to prevent pain.

Preferably wherein the local anaesthetic is lidocaine.

### BRIEF DESCRIPTION OF THE DRAWINGS

FIG. 1 is a schematic top perspective view of the skin tag remover shown in an open loop position together with a skin tag.

FIG. 2 is a schematic top perspective view of the skin tag remover shown in FIG. 1 however in a constricted position shown deployed with a skin tag.

FIG. 3 is a schematic partial enlarged view of the cord lock and cutting mechanism of the skin tag remover wherein the cutting mechanism is shown in the open position.

FIG. 4 is a schematic partial enlarged view of the cord lock and cutting mechanism shown in the cut position 161.

FIG. 5 is a schematic top plan view of the skin tag remover shown in the open loop position.

FIG. 6 is a schematic top plan view of the skin tag remover shown in the substantially constricted position.

FIG. 7 is schematic cross sectional view along section A-A of FIG. 5 showing the cord lock features, the cord channel together with the cutting mechanism.

FIG. 7A is a cross sectional view taken along section B-B in FIG. 7 showing the features of the cord lock deployed in the lock receptacle together with the lock fingers.

FIG. 8 is a schematic perspective view of a ball cord.

FIG. 9 is a schematic perspective view of a stepped cord.

FIG. 10 is a schematic perspective view of a barbed cord.

### BEST MODE FOR CARRYING OUT THE INVENTION

The present concept a skin tag remover shown generally as 100 in the drawings includes a cord housing 102, a cord 104 forming a loop 106 at a tag end 108 and is attached to a finger ring 110 at a ring end 112.

Cord housing 102 further includes a plunger 114, a cord lock 116 which is slideably received within cord housing 102.

Cord housing 102 further includes a semi-circular shaped loop receiving end 120 and also a semi-circular shaped ring receiving end 122.

Loop 106 is shown in an open loop position 124 which is surrounding a skin tag 126 proximate the tag base 118.

Referring now to FIG. 2 which shows loop 106 in a constricted position 130 wherein by using finger ring 110 one is able to pull cord 104 in the constricting direction 132 thereby pulling cord 104 through cord housing 102 thereby contracting or constricting loop 106 to the point where it strangulates tag base 118 to the point that it cuts off the blood supply to skin tag 126.

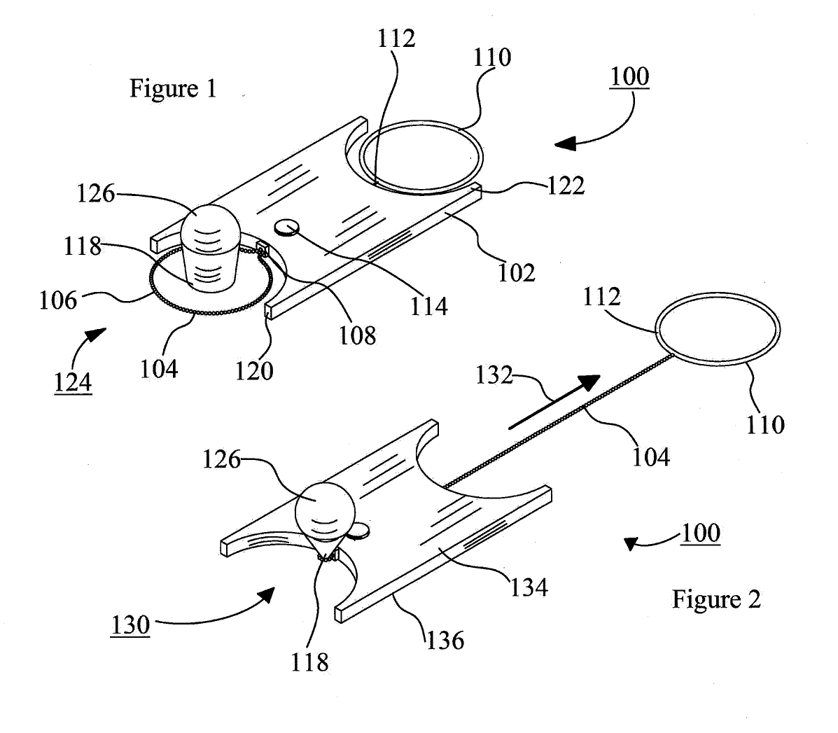

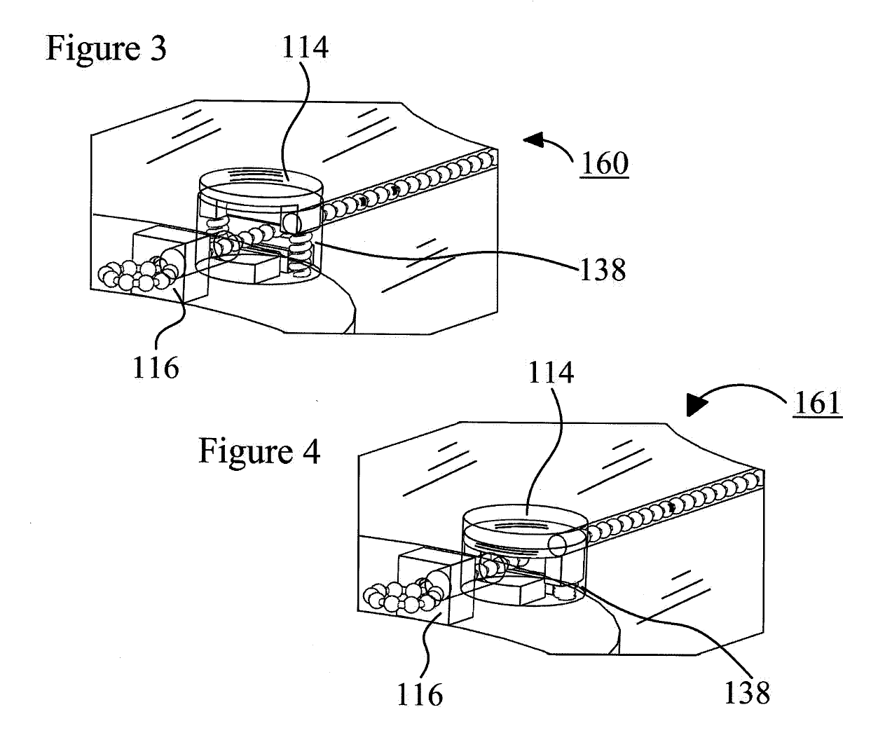

Skin tag remover 100 further includes a top side 134 and a bottom side 136. Normally in use bottom side 136 is placed onto an individual's skin in proximity to the skin tag such that skin tag 126 is surrounded by loop 106 as shown in FIG. 1.

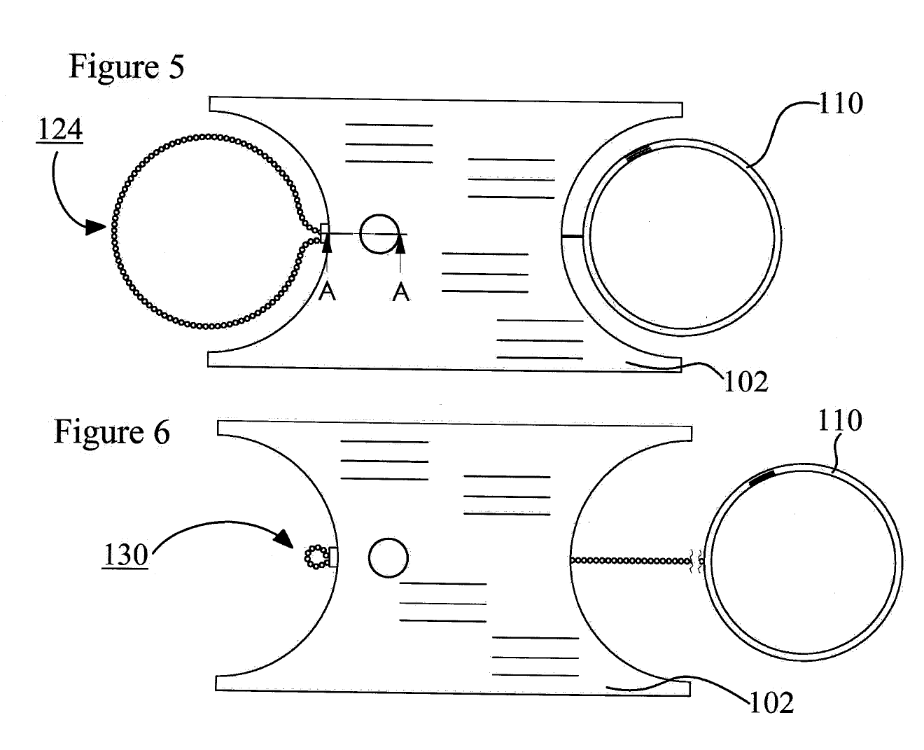

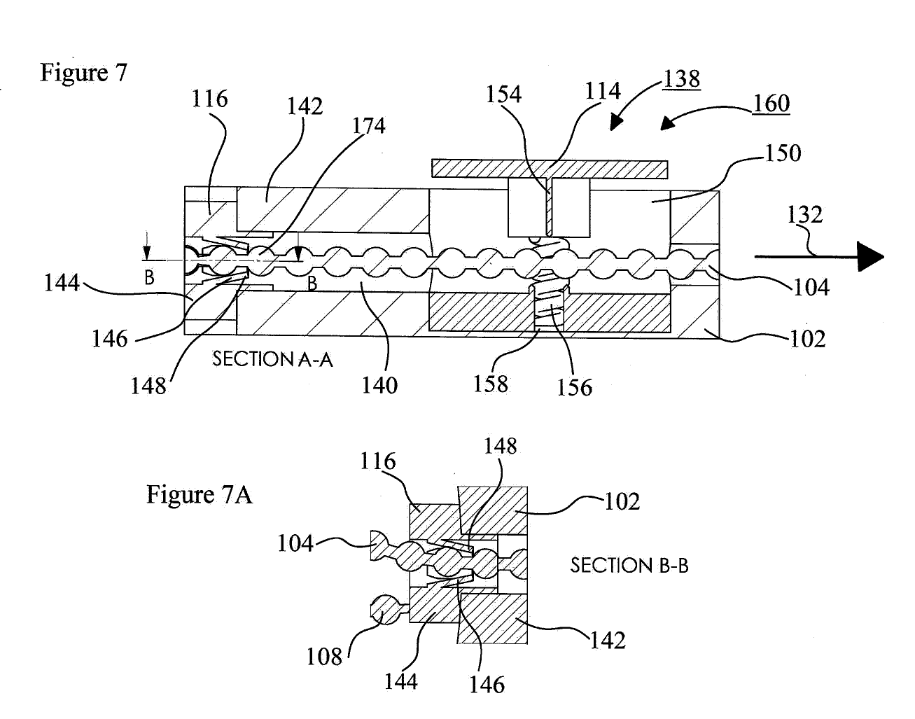

Referring now to FIGS. 7 and 7(a) which are cross sections taken along lines A-A in FIG. 5 and B-B in FIG. 7 respectively.

Referring first of all to FIG. 7 which depicts the cord channel 140 of cord housing 102 and a cord 104 lying within cord channel 140.

Near tag end 108 of cord housing 102 is a cord lock 116 which is slideably received within a lock receptacle 142.

Cord lock 116 includes a lock housing 144 and lock fingers 146 which are resiliently biased against cord 104.

Also depicted in FIG. 7 is a cutting mechanism 138 which includes plunger 114 which is connected to a blade 154 and is resiliently biased in the open position 160 with springs 156. As plunger 114 is depressed by finger pressure down through plunger cavity 150 it is guided through blade receiving guide 158.

The reader will note that as cord 104 is pulled in the constricting direction 132 through cord channel 140 defined within cord housing 102 the lock fingers 146 which are resiliently biased against cord 104 will prevent reverse movement in the opposite direction to the constricting direction 132 thereby continually locking cord 104 into position or in other words irreversibly closing off loop 106 from the open loop position 124 to the constricted position 130 as one pulls in the constricting direction 132.

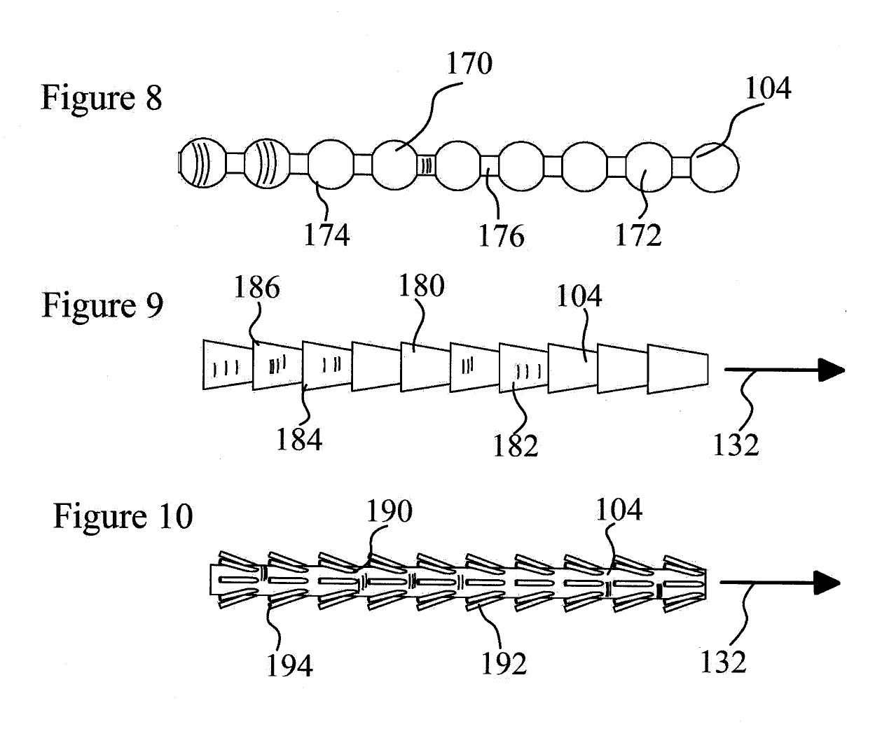

Finger lock end 148 abuts against ball protrusion 174 shown in FIG. 8 thereby preventing reversal of direction of cord 104 as it travels in the constricting direction 132 through cord channel 140 in cord housing 102.

Lock fingers 146 are resiliently biased against cord 104 and the lock fingers 146 flex overtop of ball protrusions 174 and abuts against the back end of each ball protrusion thereby locking cord 104 in its travel through cord channel 140 along the constricting direction 132.

Referring now to FIGS. 8, 9 and 10 the reader will note that there are various possibilities of cord that could be utilized with skin tag remover 100 namely ball cord 170 which is made up of ball sections 172 and transition sections 176 thereby defining ball protrusions 174.

FIG. 9 depicts cord 104 which is a step cord 180 which includes tapered elements 182 and step protrusions 184 such that the finger lock ends 148 of flexible lock fingers 146 abut against steps 186 thereby providing for the locking action.

Referring now to FIG. 10 cord 104 is shown as a barbed cord 190 which includes rearwardly projecting barbs 192 and barbed protrusions 194. In similar fashion the flexible lock fingers 146 make contact with barbed protrusions 194 thereby providing for the locking action.

The reader will note that the cords 104 depicted in FIGS. 9 and 10 namely stepped cord 180 and barbed cord 190 are directional in nature namely the tapered elements and the barbs are oriented in such a way that it allows the cord to flow through and past the cord lock 116 in the constricting direction 132 however it does not allow the reversal of this motion.

Ball cord 170 also provides for the same locking action however cord 104 is not directional in nature in that it can accomplish the same result regardless of the direction in which the cord is fed through the cord channel 140.

Cord 104 is preferably coated with an antibacterial agent to prevent infection and also with a local anaesthetic agent to prevent any expected pain. Preferably two layers are applied on antibacterial agent such as chlorhexidine and a local anaesthetic suck as lidocaine.

In Use

In order to use skin tag remover 100 the bottom side 136 of cord housing 102 is placed upon a user's skin such that loop 106 encompasses skin tag 126 when loop 106 is in the open loop position 124 shown in FIG. 1.

Subsequently finger ring 110 is urged in the constricting direction 132 such that cord 104 is pulled through cord housing 102 and along cord channel 140 to the point where loop 106 is placed in the constricted position wherein the tag base 118 of skin tag 126 is strangulated to the point where the blood supply of the skin tag 126 cut off.

At this point one is able to depress plunger 114 and cut cord 104 with blade 154 thereby separating the ring end 112 of cord 104 from the tag end 108 of cord 104 facilitating removal of the cord housing 102 from the skin.

The user is left with the cord 104 in the constricted position 130 around the skin tag 126 together with the cord lock 116 which slideably is removed from cord housing 102 by moving the entire cord housing 102 also in the constricting direction 132 away from the skin tag 126.

In this manner after a few days of leaving the cord 104 in the constricted position 130 the blood supply to skin tag 126 is cut off and the skin tag will naturally die and fall off the skin together with cord 104 thereby achieving skin tag removal.

It should be apparent to persons skilled in the arts that various modifications and adaptation of this structure described above are possible without departure from the spirit of the invention the scope of which defined in the appended claim.

I claim:

1. A skin tag remover which includes:
a) a cord formed into a loop at a tag end, the loop for placing around a skin tag;
b) a cord housing for receiving a portion of the cord there through;
c) a finger ring attached to a ring end of the cord; such that pulling on the finger ring tightens the loop around the skin tag and places the cord into a constricted position;
d) a means for locking the cord in a constricted position.

2. The skin tag remover claimed in claim 1 further including a means for cutting the cord such that the loop is separated from the finger ring.

3. The skin tag remover claimed in claim 1 wherein the locking means includes a lock housing slide-able received within a lock receptacle, such that the cord lock is slideably detachable from the cord housing.

4. The skin tag remover claimed in claim 3 wherein the locking means includes lock fingers which abut against the cord protrusions allowing movement of the cord in only the constricting direction thereby preventing the cord from loosening from around the skin tag.

5. The skin tag remover claimed in claim 2 wherein the cutting means includes:
a) a blade biased in a normal open position above the cord;
b) a plunger connected to the blade for urging downwardly with finger pressure thereby cutting the cord;
c) the plunger received within a plunger cavity defined within the cord housing.

6. The skin tag remover claimed in claim 5 wherein the cutting means includes a blade receiving guide for receiving the blade therein in a closed position.

7. The skin tag remover claimed in claim 1 wherein the cord housing includes:
a) a cord channel guiding the cord through the cord housing.

8. The skin tag remover claimed in claim 1 wherein the cord housing includes a semi-circular ring receiving end and a semi-circular loop receiving end for mounting the loop and finger ring in position when placed on the skin.

9. The skin tag remover claimed in claim 1 wherein the cord is selected from among a ball cord, a stepped cord and a barbed cord.

10. The skin tag remover claimed in claim 1 wherein the cord is coated with an antibacterial agent to prevent infection.

11. The skin tag remover claimed in claim 10 wherein antibacterial coating is chlorhexidine.

12. The skin tag remover claimed in claim 10 wherein the cord is further coated with a local anaesthetic to prevent pain.

13. The skin tag remover claimed in claim 12 wherein the local anaesthetic is lidocaine.
